(19) United States
(12) Patent Application Publication
(10) Pub. No.: US 2012/0039927 A1
(43) Pub. Date: Feb. 16, 2012

(54) USE OF EXTRACTS OF PEZIZACEAE IN THE PREVENTION AND/OR TREATMENT OF SENILE CATARACTS

(76) Inventor: Arvin Morgan, West Park, FL (US)

(21) Appl. No.: 13/205,693

(22) Filed: Aug. 9, 2011

Related U.S. Application Data
(60) Provisional application No. 61/372,137, filed on Aug. 10, 2010.

(51) Int. Cl. A61K 36/062; A61P 27/12

(52) U.S. Cl. 424/195.15

(57) ABSTRACT

The invention provides methods for treating and/or preventing the recurrence of a senile cataract, and/or for inhibiting the development of a senile cataract and/or an incipient senile cataract, in a human or animal subject. The invention further provides nutraceutical compositions suited for the same, derived from an extract of truffles (Pezizaceae). The compositions comprise the extract in a therapeutically sufficient amount for the treatment and/or prophylaxis of a senile cataract in a human and/or animal subject.

### CROSS-REFERENCE TO RELATED APPLICATIONS

This application claims the benefit of prior U.S. Provisional Patent Application Ser. No. 61/372,137, filed Aug. 10, 2010.

### BACKGROUND OF THE INVENTION

1. Field of the Invention

The present invention relates to the field of medicine, and in particular, to the field of ophthalmology. More specifically, the present invention relates to novel non-surgical methods, having both medical and veterinary application, for the prevention and/or treatment of a particular class of cataracts; the invention also relates to fungal formulations that find use in such methods, and which were previously unknown for such use.

2. Technical Problem Addressed

The lens of the eye is a transparent, bi-convex body, which functions as a refractive device to focus light onto the retina. A cataract is an opacity of the lens that adversely affects visual acuity. Cataracts are the leading cause of blindness in the world and incidence of cataract-induced blindness is expected to increase, especially in developing countries. Cataracts can appear at all ages, but are most commonly associated with aging adults—approximately 90% of adults 70 years and older experience symptoms of cataracts. However, age-related or senile cataracts also occur in the later lives of other animals, including species in other Families of Order Primates, as well as species from other Orders and Families of Class Mammalia, such as mice and rats (Order Rodentia), dogs (Family Carnidae), cats (Family Felidae), horses (Family Equidae), cows (Family Bovidae) and other higher mammals. Species from other non-mammalian Classes, such as birds (Class Ayes), are also affected by senile cataracts.

The precise cause of cataract formation is unclear. Alteration in cell structure, formation of water pockets, and accumulation of high molecular weight proteins are but a few of the changes in lens architecture associated with cataract formation. Loss of transparency has been linked to formation of opaque fibers in the layers of the lens, as well as loss of transparency of previously clear fibers due to protein damage. Accumulation of extracellular materials and pigment also result in loss of transparency and scattering of light.

Symptoms of cataracts include blurred and double vision, color confusion and reduced vision at night and in low light. As vision gradually deteriorates over time, affected individuals lose the ability to accomplish everyday tasks. Decreased vision results in difficulty in reading and watching television. Many have difficulty driving, especially at night. As such, many patients must alter their lifestyles and give up much of their independence. Cataract patients also have an increased risk of injury due to impaired vision.

Presently, cataract surgery is the only generally accepted treatment for cataracts (senile or otherwise), and for reasons that are self-evident, is performed almost exclusively only in humans. In cataract surgery, the affected lens is removed (excised) and replaced by a synthetic (generally plastic or silicone) intraocular lens. While this procedure typically provides human patients with at least 20/40 vision, it is not without its drawbacks.

Many complications can arise during and after surgery. Tearing of the capsule of the lens can cause the posterior capsule to rupture, thereby losing the lens to the vitreous cavity. Hemorrhage during surgery can stimulate inflammation and accelerate opacification of the capsule. Epithelial cells can invade the wound into the cornea, iris, and lens capsule, thereby causing uveitis and corneal decompensation. Retinal detachment is also a risk following cataract surgery. A rise in intraocular pressure is common after cataract surgery and causes glaucoma-like symptoms. Furthermore, some human patients may not be candidates for cataract surgery, due to either a pre-existing medical condition or an unwillingness to undergo an ocular surgery, and animals suffering from cataracts are rarely candidates for cataract surgery, since unlike human subjects, in most cases the extracted lens would not be replaced with a synthetic lens implant, so the animal would remain virtually blind in any event, and also due to the relatively high cost of such veterinary procedures.

Accordingly, a method by which cataracts could be treated, in both humans and animals, by a non-surgical process would be a useful addition to ophthalmic medicine. Yet, despite much study of the cause and treatment of cataracts, a clinically useful, non-surgical treatment that retards the development of senile cataracts has thus far eluded researchers.

It is therefore the principal object of this invention to provide a clinically useful, non-surgical treatment, having virtually no side effects or complications, to reduce the symptoms, or to prevent the development, of senile cataracts in the lens of the eye of a human or animal.

It is another object of the present invention to provide a clinically useful, non-surgical treatment that will significantly retard the rate of development of senile cataracts, and thereby eliminate the need for many surgical cataract extractions.

It is yet another object of the present invention to provide a fungal medicament having prophylactic and/or therapeutic properties with respect to the formation and/or progression of senile cataracts in the lens of the eye of a human or an animal.

3. Description of Related Art

Truffles, which belong to the class of ascomycetes, order of Pezizales, have been appreciated as excellent edible fungi for many centuries and are of outstanding importance due to their special aroma. Truffles are underground fungi growing in symbiosis with certain plants and forming tuber-like fruiting bodies.

Truffles or their extracts have been reported to possess anti-inflammatory, immunosuppressor, antimutagenic, anticarcinogenic, antioxidant, antiradical, antimicrobial and hepatoprotective properties, as well as a steroidal glucoside with polyhydroxy ergosterol nucleus (tuberoside). Truffles or their extracts have also been reported to be used as a nourishing and invigorating preparation for convalescents, as an aphrodisiac, as an ingredient in the manufacture of a cosmetic preparation, and as a medicine to treat diabetes, skin diseases, stomach ailments, open cuts, bacterial eye infections and eye pain. However, prior to the development of the present invention, no use of any truffle species, or of any extract or component thereof, to reduce or eliminate the lens opacity that typifies any type of cataract, has ever been recorded.

### BRIEF SUMMARY OF THE INVENTION

The present invention resides in the surprising discovery, determined through preliminary in-vivo experiments described hereinafter, that the use of an extract of truffles (Pezizaceae) effectively reduces or eliminates the lens opacity that typifies senile cataracts. It has also been found that the continued use of Pezizaceae effectively prevents the recurrence of senile cataracts in previously affected eyes, and also prevents the occurrence of senile cataracts in eyes at risk for developing such a condition. Methods of treating senile cataracts are provided, the methods employing an aqueous extract of Pezizaceae to treat a patient in need of treatment for senile cataracts. An ophthalmically stable aqueous solution appropriate for dispensing to the eye, for the prevention and/or treatment of senile cataracts, is also provided.

The aqueous solution of the invention includes an extract of Pezizaceae in a therapeutically sufficient amount over time so as to prevent or retard the development of a cataract in a human or animal subject to which the composition is administered. The composition may be a topical preparation or other dosage unit form suitable for instillation in the human or animal eye topically, systemically, or intraocularly. Topical dosage forms may include liquid eye drop preparations or ointments that may be instilled externally to the eye, or adsorbed into a material such as a soft contact lens or a collagen corneal shield. On the other hand, intraocular administration may take such forms as periocular injection or intraocular instillation (for example, by implantation of an intraocular reservoir).

The methods of the invention include methods for inhibiting or preventing the development of a senile cataract, or for inhibiting the progression of an incipient senile cataract, in a human or animal subject, which methods comprise administering to such a human or animal subject an aqueous extract of Pezizaceae in a therapeutically sufficient amount to prevent or retard the development of a senile cataract in such a subject.

In one embodiment of the invention, an extract of Pezizaceae is applied to the affected eye which treats an existing senile cataract. In another embodiment, the continued use of such an extract effectively prevents a senile cataract from recurring in the affected eye. In yet another embodiment of the invention, such an extract is used as a prophylactic treatment to reduce the risk of developing a senile cataract.

In a further embodiment of the invention, such an extract is used in conjunction with ocular surgery, e.g., a patient with a severe senile cataract may first be treated with such an extract to diminish the severity of the cataract, which is subsequently removed using conventional ocular surgical techniques. In yet another embodiment of the invention, such an extract may be used post-operatively.

These and other objects, aspects, features and advantages of the present invention will become more apparent from, and will be understood more clearly by reference to, the following detailed description of the presently most preferred embodiment thereof.

### DETAILED DESCRIPTION OF THE PREFERRED EMBODIMENT

The preferred and other embodiments of the present invention will now be further described. Although the invention will be illustratively described hereinafter with reference to a fungal nutraceutical formulation comprising an aqueous extract of truffles (Pezizaceae), it should be understood that the invention is not limited to aqueous extracts, but extends to formulations (and to methods utilizing them) that are prepared using other available extraction techniques, provided that each one yields the active components which are present in the Pezizaceae and that exhibit the salutary bio-efficacious properties of the present invention as disclosed herein.

The present invention resides in the surprising finding that an extract of Pezizaceae can be formulated into compositions that are useful for preventing the formation of senile cataracts, or for treating such cataracts, especially by slowing their progression, in animals, including humans.

In a preliminary in-vivo experiment conducted by applicant, which will be described in further detail hereinafter, it was discovered that an extract of Pezizaceae was useful in ameliorating the effects of senile cataracts in an animal, specifically, a canine. The results of this experiment prompted the applicant to conduct an additional in-vivo experiment, also to be described below, which showed that such extracts were also useful in preventing and/or treating senile cataracts in a human subject. Thus, while at present this invention finds use in the prevention and/or treatment of senile cataracts, further research may reveal that such extracts may be useful in treating non-senile cataracts, such as diabetic cataracts, steroid-induced cataracts or cataracts induced by exposure to chemicals or to radiation.

In a preferred embodiment of the invention, an extract is prepared from truffle species within the Pezizaceae family, including but not limited to Terfezia claveryi Chatin, Terfezia boudieri Chatin, Terfezia arenaria (Moris) Trappe, Terfezia leptoderma Tul., Tirmania nivea (Desf.) Trappe, Tirmania pinoyi (Maire) Malencon and mixtures thereof, particularly Terfezia claveryi Chatin, as described in further detail below. In alternative embodiments, other truffle species within the Pezizalean families Glaziellaceae, Discinaceae-Morchellaceae, Helvelleceae, Tuberaceae and Pyronametaceae, particularly Tuberaceae, may be used in preparing the extract, as it is believed that whatever the active components may be, which are present in truffle species within the Pezizaceae family and which exhibit the salutary properties of the present invention, may be found in truffle species within other Pezizalean families as well.

In one embodiment of the invention, the extract is prepared by a conventional aqueous extraction method, that is, by mixing Pezizaceae with water. Specifically, the aqueous solution is prepared by the following steps:

a) Providing Pezizaceae.

b) Rinsing the Pezizaceae with water.

c) Soaking the Pezizaceae in water.

d) Crushing the Pezizaceae, preferably in a gentle manner, to produce a mixture.

e) Producing a filtrate by filtering the mixture to remove particulate matter.

f) Optionally, concentrating the filtrate by removing water.

In this embodiment of the invention it is contemplated that the Pezizaceae may be rinsed and soaked in any water of suitable purity. For example, washing may be carried out in distilled or deionized water. Preferably, the water is sterilized (e.g. by either heat or filter sterilization) prior to use.

According to one aspect of the present embodiment the Pezizaceae may be crushed by any suitable means. Exemplary methods for crushing the Pezizaceae to produce the mixture, which are contemplated for use according to this aspect of the invention, include but are not limited to smashing the Pezizaceae using a mortar and pestle and pressing the Pezizaceae through a sieve.

In order to separate the particulate matter from the mixture the mixture may be filtered using any means which is compatible with the invention. Such means may include but is not limited to filtering through a small mesh sieve, filtering through gauze or a paper filter or filtration through any other commercially available filter.

Following filtration the filtrate, constituting the aqueous extract, may then be used in accordance with the invention, although it is expected that the aqueous extract will have maximum potency immediately after its preparation, and that the effectiveness of the aqueous extract will diminish over time. If the aqueous extract is to be used directly, for topical instillation into the eye in liquid form, that is, as eye drops, then the extract solution should be shaken prior to use.

In other embodiments of the invention, the aqueous extract may, optionally, be concentrated by conventional methods before further use is made of it in accordance with the invention. In still other embodiments, Pezizaceae or its aqueous extract is extracted with an organic solvent to further separate the active components from non-active components. Such solvents may include alcohols such as methanol, ethanol or isopropanol, ketones such as acetone or methyl-ethyl ketone, esters such as ethyl acetate or butyl acetate, halogenated solvents such as methylene chloride or chloroform or hydrocarbon solvents such as hexane or toluene. In yet other embodiments, the individual components of the extract may be isolated using conventional chromatographic techniques upon conventional supports such as silica, alumina, size-exclusion and ion-exchange resins. In further embodiments, the individual components of the extract may be separated using distillation, crystallization and/or chemical derivatization. Such techniques are well known to those having skill in the chemical arts.

The nutraceutical compositions of the present invention can be formulated for prophylactic and/or therapeutic administration by various methods that are well known in the ophthalmic arts, maintaining required sterility and osmolarity. For example, in another embodiment, the aqueous extract, its concentrate or purified components thereof may be mixed with conventional pharmaceutically acceptable excipients designed to increase the stability of the extract, or of its concentrate or its purified components, so as to facilitate application to the eye, or to prolong the residence of the extract, its concentrate or its purified components upon the ocular surface. Such excipients are well known to those having skill in the ophthalmic arts. The extract, its concentrate or its purified components may be applied in conventional pharmaceutically acceptable vehicles such as a solution.

As indicated above, topical instillation in the eye is the preferred method of administration, most preferably in the form of liquid eye drops, although in addition to the alternative embodiment mentioned above of being administered topically combined with an ophthalmic ointment or lubricant, the aqueous extract, its concentrate or purified components thereof can also alternatively be administered by being adsorbed into a soft contact lens (e.g., that marketed by Johnson & Johnson under the trade name “Accuvue Advance”) or a dissolvable collagen corneal shield.

The nutraceutical compositions of the present invention can alternatively be administered either systemically or by intraocular means, although at present these administration routes are less preferred. Regarding systemic administration, that is, oral or parenteral administration, the aqueous extract, its concentrate or purified components thereof may be incorporated into tablets, pills, capsules, etc., wherein the extract, its concentrate or purified components thereof is/are dispersed in one or more pharmaceutically acceptable, preferably biodegradable, carriers, and wherein the delivery system may also include one or more of the following: binders, excipients, lubricants, glidants and/or sweetening agents.

Regarding intraocular administration, that is, direct infusion into the eyeball, the aqueous extract, its concentrate or purified components thereof can be administered via injection, or by other delivery routes known in the ophthalmic arts that are applicable to administer a drug to the eye. These administration routes can be advantageous when direct treatment of the ocular lens, or the provision of a continuous supply of the aqueous extract, its concentrate or purified components thereof to the eye may be required. However, as it is expected that discontinuous treatment over time will be the most effective form of therapy, and since, at least for human subjects, a non-invasive administration method that also admits of self-administration may be preferable, preparations which can be administered topically, particularly as liquid eye drops using a conventional eye dropper, are most preferred.

When administered in that form, as a non-concentrated aqueous extract of Pezizaceae which has not been further purified or further extracted with any organic solvents, the preferred dosage is one drop (equivalent to 50 .mu.l) in each affected eye, 3 times per day, over the course of four to six weeks. It has been found that for such a dosage regimen, the aqueous extract should be replaced with a freshly-prepared aliquot at least once every week, and that any remaining unused extract in the previous aliquot should not be used. It is also preferable that each aliquot of the aqueous extract be kept refrigerated when not in use.

### EXAMPLES

The following working examples illustrate both the manner in which such an aqueous extract of Pezizaceae in accordance with the present invention has been prepared and used in both human and animal subjects suffering from at least one senile cataract, and the experimental results obtained, which demonstrate the efficacy of the invention. However, the following examples should not be construed as in any way limiting its scope.

### Example 1

### Crude Extract From Pezizaceae

Aqueous Extraction of Pezizaceae: Fresh truffles of the species Terfezia claveryi Chatin were collected in Damascus, Syria. 1 truffle (50 grams) was washed three times with distilled water. Next the truffle was cut into small pieces and soaked in distilled water (1:3) for 24 hours. The pieces of truffle were then gently smashed (while still in the water), using a mortar and pestle, to prepare a mixture of truffle extract and crushed truffle tissue. The mixture was then filtered through gauze to remove the large particulate matter. An aliquot of the filtered extract was removed and dispensed into a sterile dropper bottle.

### Example 2

A 12 year old female dog observed as having greatly impaired vision due to cataracts was treated with the eye drop formulation prepared in Example 1. In particular, the eye drop formulation of Example 1 was administered to each eye of the animal subject, three times per day, for three weeks. After three weeks of treatment the size of each cataract was substantially reduced. In addition to the actual physical reduction in the size of the cataract, the visual behavior of the dog was substantially improved. For example, prior to the treatment with the eye drops, the subject was observed squinting her eyes and constantly bumping into objects, as well as being reluctant to climb stairs and jump up onto furniture as she normally did. After completion of the treatment regimen, the dog ceased squinting her eyes and bumping into objects, and was no longer reluctant to climb stairs or jump up onto furniture, and could navigate generally without any observed visual impairment.

### Example 3

A 65 year old African-American male having cataracts in each eye and suffering from substantial vision impairment was treated with the eye drop formulation prepared in Example 1. In particular, the eye drop formulation of Example 1 was given to the patient, who thereafter self-administered one drop in each eye three times per day, for four weeks. The patient was given, and thereafter used, a freshly-prepared aliquot of the aqueous extract once per week, and each aliquot was kept refrigerated when not in use. After four weeks of treatment the patient's cataracts were substantially eliminated from each of his eyes. Furthermore, each eye was shiny and transparent. In addition to the actual physical disintegration of the cataract, the vision of the patient was substantially improved. For example, prior to the treatment with the eye drops, the patient could not see well enough to read the newspaper or drive an automobile at night. After completion of the treatment regimen, the patient could see well enough to read the newspaper and drive at night without significant difficulty. Moreover, the patient suffered no adverse reaction from the four week treatment regimen.

Based on prior experimental results, it is believed that the present invention comprises treatment methods and compositions that can ameliorate the effects, and/or inhibit the development, of senile cataracts in an animal subject, for example, a dog, a cat, a horse, a cow, a mouse, a rat or a human. As to human subjects in particular, it should be understood that, when used prophylactically, although any patient in a demographic group at significant risk for senile cataracts can be treated (for example, humans over the age of 50), subjects can also be selected using more specific criteria. For example, patients who have biomicroscopic clinical evidence of an incipient senile cataract, or patients who have biomicroscopic evidence of a senile cataract combined with a decrease in visual acuity, can be selected for treatment.

The present invention is useful in the treatment and/or prophylaxis of senile cataracts, and therefore finds industrial applicability in human and veterinary medicine.

While there has been described what are at present considered to be the preferred embodiments of the present invention, it will be apparent to those skilled in the art that the embodiments described herein are by way of illustration and not of limitation. Therefore, it is to be understood that various changes and modifications may be made in the embodiments disclosed herein without departing from the true spirit and scope of the present invention, as set forth in the appended claims.

What is claimed is:

1. A method of treating a senile cataract in a human or veterinary patient, comprising administering a therapeutically effective amount of an extract of truffles (Pezizaceae) to a human or veterinary patient in need of such treatment.

2. A method in accordance with claim 1 wherein said extract is an aqueous extract and is prepared from Pezizaceae selected from the group consisting of Terfezia claveryi Chatin, Terfezia boudieri Chatin, Terfezia arenaria (Moris) Trappe, Terfezia leptoderma Tul., Tirmania nivea (Desf.) Trappe, Tirmania pinoyi (Maire) Malencon and mixtures thereof.

3. A method in accordance with claim 2 wherein said extract is prepared from Terfezia claveryi Chatin.

4. A method in accordance with claim 1 wherein said extract is administered topically, systemically or intraocularly.

5. A method in accordance with claim 4 wherein said extract is administered topically to an afflicted eye of the patient in the form of liquid eye drops.

6. A method in accordance with claim 5 wherein said patient is human and said extract is administered in an amount comprising substantially 0.15 ml daily, for a period of four to six weeks.

7. A method in accordance with claim 6 wherein said extract is an aqueous extract prepared from Pezizaceae selected from the group consisting of Terfezia claveryi Chatin, Terfezia boudieri Chatin, Terfezia arenaria (Moris) Trappe, Terfezia leptoderma Tul., Tirmania nivea (Desf.) Trappe, Tirmania pinoyi (Maire) Malencon and mixtures thereof.

8. A method in accordance with claim 7 wherein said extract is administered together with one or more pharmaceutically acceptable excipients and/or carriers.

9. A method for inhibiting or preventing the development of a senile cataract or inhibiting the progression of a senile cataract or an incipient senile cataract in a subject, the method comprising the step of administering to a subject in need of such treatment a therapeutically effective amount of an extract of Pezizaceae, together with one or more therapeutically acceptable additives.

10. A method in accordance with claim 9 wherein said extract is an aqueous extract and is prepared from Pezizaceae selected from the group consisting of Terfezia claveryi Chatin, Terfezia boudieri Chatin, Terfezia arenaria (Moris) Trappe, Terfezia leptoderma Tul., Tirmania nivea (Desf.) Trappe, Tirmania pinoyi (Maire) Malencon and mixtures thereof.

11. A method in accordance with claim 10 wherein said extract is prepared from Terfezia claveryi Chatin.

12. A method in accordance with claim 11 wherein said extract is administered topically, systemically or intraocularly.

13. A method in accordance with claim 12 wherein said extract is administered topically to an afflicted eye of the subject in the form of liquid eye drops.

14. A method in accordance with claim 13 wherein said subject is a human being and said extract is administered in an amount comprising substantially 0.15 ml daily, for a period of four to six weeks.

15. A method in accordance with claim 14 wherein said extract is an aqueous extract prepared from Pezizaceae selected from the group consisting of Terfezia claveryi Chatin, Terfezia boudieri Chatin, Terfezia arenaria (Moris) Trappe, Terfezia leptoderma Tul., Tirmania nivea (Desf.) Trappe, Tirmania pinoyi (Maire) Malencon and mixtures thereof.

16. A non-surgical method of alleviating a senile cataract in the eye of a human or animal subject, comprising the step of administering a fungal medicament to alleviate the senile cataract, the medicament comprising an extract prepared from Pezizaceae selected from the group consisting of Terfezia claveryi Chatin, Terfezia boudieri Chatin, Terfezia arenaria (Moris) Trappe, Terfezia leptoderma Tul., Tirmania nivea (Desf.) Trappe, Tirmania pinoyi (Maire) Malencon and mixtures thereof.

17. A method in accordance with claim 16 wherein said extract is an aqueous extract and is prepared from Terfezia claveryi Chatin.

18. A method in accordance with claim 17 wherein said subject is human.

19. A method in accordance with claim 16 wherein said extract is administered topically, systemically or intraocularly.

20. A method in accordance with claim 19 wherein said extract is administered topically to an afflicted eye of the subject in the form of liquid eye drops.

21. A method in accordance with claim 20 wherein said subject is a human being and said extract is administered in an amount comprising substantially 0.15 ml daily, for a period of four to six weeks.

22. A method in accordance with claim 21 wherein said extract is an aqueous extract prepared from Terfezia claveryi Chatin.

23. A method for reducing lens opacity in an eye of a human or non-human subject having or being susceptible to a senile cataract, the method comprising the step of administering to the afflicted eye a composition comprising an extract of Pezizaceae in an amount therapeutically sufficient to prevent or retard the development of a senile cataract in a subject to whom the composition is administered.

24. A method in accordance with claim 23 wherein said subject is a human subject and said extract is an aqueous extract prepared from Terfezia claveryi Chatin.

25. A method in accordance with claim 24 wherein said extract is administered topically in the form of liquid eye drops.

26. A method of treating a disorder characterized by opacity in the lens of an eye of an animal caused by a senile cataract, the method comprising administering to an animal having said disorder an amount of an extract from Pezizaceae comprising substantially 0.15 ml daily, for a period of four to six weeks.

27. A method in accordance with claim 26 wherein said animal is a human subject, and wherein said extract is an aqueous extract prepared from Terfezia claveryi Chatin.

28. A method in accordance with claim 27 wherein said extract is administered topically to the afflicted eye in the form of liquid eye drops.

29. A composition useful for the treatment of a senile cataract in a human or animal patient comprising an extract obtained from Pezizaceae, wherein the composition comprises said extract and one or more physiologically acceptable additives.

30. A composition in accordance with claim 29 wherein said extract is an aqueous extract prepared from Terfezia claveryi Chatin.

31. A composition in accordance with claim 30 wherein said one or more physiologically acceptable additives comprise one or more pharmaceutically acceptable excipients and/or carriers.

32. A method of treating a senile cataract comprising the step of administering to a human or animal patient a therapeutically effective amount of the composition as claimed in claim 29.

33. A composition for the treatment of a senile cataract in an animal subject comprising an extract of Pezizaceae in a delivery system, wherein the extract comprises an aqueous extract obtained from Pezizaceae selected from the group consisting of Terfezia claveryi Chatin, Terfezia boudieri Chatin, Terfezia arenaria (Moris) Trappe, Terfezia leptoderma Tul., Tirmania nivea (Desf.) Trappe, Tirmania pinoyi (Maire) Malencon and mixtures thereof, and wherein the composition is administered to a subject in need of treatment of a senile cataract.

34. A composition in accordance with claim 33 wherein said animal subject is a mammal.

35. A composition in accordance with claim 34 wherein said mammalian subject is a human being.

36. A composition comprising an extract of Pezizaceae in a therapeutically sufficient amount to prevent or retard the development of a senile cataract in an animal subject to which the composition is administered.

37. A composition in accordance with claim 36 wherein said extract is an aqueous extract prepared from Terfezia claveryi Chatin.

38. A composition in accordance with claim 37 further comprising one or more pharmaceutically acceptable excipients and/or carriers.

39. A composition for retarding the development of a senile cataract in a subject, comprising an extract of Pezizaceae in a therapeutically sufficient amount to prevent or retard the development of a senile cataract in a subject to whom the composition is administered.

40. A composition in accordance with claim 39 wherein said extract is an aqueous extract prepared from Terfezia claveryi Chatin.

41. A composition in accordance with claim 40 further comprising one or more pharmaceutically acceptable excipients and/or carriers.

42. A composition in accordance with claim 41 wherein said subject is a mammalian subject.

43. A composition in accordance with claim 42 wherein said mammalian subject is a human being.

44. A nutraceutical mixture containing an extract of Pezizaceae in admixture with suitable excipients and/or carriers, wherein the extract is present in an effective proportion such that, when administered to a subject in a therapeutically effective amount, the mixture is effective to prevent or retard the development of a senile cataract in a subject to whom the mixture is administered.

45. A mixture in accordance with claim 44 wherein said extract is an aqueous extract prepared from Pezizaceae selected from the group consisting of Terfezia claveryi Chatin, Terfezia boudieri Chatin, Terfezia arenaria (Moris) Trappe, Terfezia leptoderma Tul., Tirmania nivea (Desf.) Trappe, Tirmania pinoyi (Maire) Malencon and mixtures thereof.

46. A mixture in accordance with claim 45 wherein said extract is prepared from Terfezia claveryi Chatin.

47. A mixture in accordance with claim 46 wherein said subject is a human subject.
